(19) United States
(12) Patent Application Publication
(10) Pub. No.: US 2025/0143824 A1
(43) Pub. Date: May 8, 2025

(54) SAFETY MODES FOR MEDICAL DEVICES

(71) Applicant: Canon U.S.A., Inc., Melville, NY (US)

(72) Inventor: Zachary Hamilton Haubert, Boston, MA (US)

(21) Appl. No.: 18/833,356

(22) PCT Filed: Jan. 26, 2023
(86) PCT No.: PCT/US23/61365

§ 371 Date: Jul. 25, 2024

Related U.S. Application Data
(60) Provisional application No. 63/303,859, filed on Jan. 27, 2022.

(51) Int. Cl. A61B 34/00; A61B 34/30; A61M 25/01

(52) U.S. Cl. CPC A61B 34/71; A61B 2034/301; A61M 25/0138; A61M 25/0147

(57) ABSTRACT

A continuum robot having at least two independently manipulateable bendable section for advancing the robot through a passage, without contacting fragile elements within the passage, wherein the robot incorporates a system, method and apparatus for rapid removal of the catheter in emergency situations. The continuum robot including a first bending section having a distal end and a proximal end wherein the first bending section is bent by at least one wire; a driver that drives the at least one wire; and a controller that controls a driving amount of the wire, wherein, the driver further comprises a safe mode that disengages the driver from the at least one wire.

### CROSS REFERENCE TO RELATED PATENT APPLICATIONS

This application claims priority from U.S. Provisional Patent Application No. 63/303,859, filed on Jan. 27, 2022, in the United States Patent and Trademark Office, the disclosure of which is incorporated by reference herein, in its entirety.

### FIELD OF THE DISCLOSURE

The present disclosure relates generally to medical devices and, more particularly to a steerable continuum robot (also referred to as ‘snake’ or ‘snake system’) applicable to guide interventional tools and instruments, such as endoscopes and catheters, in medical procedures, and safety procedures and mechanisms associated with the steerable robot.

### BACKGROUND OF THE DISCLOSURE

A steerable continuum robot or snake includes a plurality of bending sections having a flexible structure, wherein the shape of the continuum robot is controlled by deforming the bending sections. The snake mainly has two advantages over a robot including rigid links. The first advantage is that the snake can move along a curve in a narrow space or in an environment with scattered objects in which the rigid link robot may get stuck. The second advantage is that it is possible to operate the snake without damaging surrounding fragile elements because the snake has intrinsic flexibility.

Existing snake system, as seen in United States Publication No. 2019/0015978, can contain four operational modes (Follow-The-Leader “FTL”; reverse-FTL, Target, and Backdrive) which allow the user to utilize the catheter for certain procedural situations. This involves insertion, navigation, tip positioning, and relaxing of the drive wires for better steer-ability.

Aside from the operational modes, a workflow exists in the attachment of the catheter to the actuation unit. For instance, the catheter hub is first attached to the base of the actuation unit, then the individual catheter drive wires are connected to their respective actuators within the actuation unit. Once both connections are made, the operational modes can be carried out. To remove the catheter, first the wires must be disconnected, then the hub.

However, in particular cases, the Snake procedure may require the emergency removal of a steerable catheter attached to an actuation unit with a robot controller. When the physician identifies a situation to prompt emergency removal, the physician would need to disconnect the driving forces from the actuation unit to the steerable catheter as soon as possible and remove the steerable catheter from a patient safely.

However, the safe system procedure to realize this emergency removal with the steerable catheter is not disclosed in the existing literature. Which is the aim of the subject disclosure.

The system will require certain inputs from the user, as well as outputs to allow the emergency removal procedure to be carried out by the user.

This emergency workflow currently does not exist in the Snake system. In the current state, emergency removal can be potentially harmful to the patient as the system has no way of knowing there is an emergency. The user can potentially operate the actuator while the catheter has been disconnected. There is also no convenient or safe mechanism for releasing the catheter drive wires, which is necessary in removing the catheter from the cavity.

### SUMMARY

Thus, to address such exemplary needs in the industry, the presently disclosed apparatus teaches a robotic apparatus comprising A robotic apparatus comprising a continuum robot including a plurality of bending sections including a distal bending section and a proximal bending section wherein each of the bending sections are bent by at least one wire, as well as a driver that drives the wire, and a controller that controls a driving amount of the wire, wherein, the controller further comprises a safe mode that disengages the driver from the at least one wire.

In other embodiment, the subject disclosure teaches a robotic apparatus comprising a continuum robot including a plurality of bending sections including a distal bending section and a proximal bending section wherein each of the bending sections are bent by at least one wire, and includes a driver that drives the wire, a controller that controls a driving amount of the wire, and a base affixed to the continuum robot and capable of moving the continuum robot, wherein, the controller further comprises a safe mode that disengages the driver from the at least one wire.

In yet another embodiment, the innovation teaches a continuum robot control means comprising a continuum robot including a plurality of bending sections including a distal bending section and a proximal bending section wherein each of the bending sections are driven by at least one wire, and further includes a driving means that drives the wire, and a control means that controls a wire driving amount from a bending angle and a rotational angle of the continuum robot, wherein the control means includes a safe mode that disengages the driver from the at least one wire.

Finally, the subject disclosure also teaches a continuum robot control means comprising a continuum robot including a plurality of bending sections including a distal bending section and a proximal bending section wherein each of the bending sections are driven by at least one wire, as well as a driving means that drives the wire, and a control means that controls a wire driving amount from a bending angle and a rotational angle of the continuum robot, and the control means also controls a base affixed to the continuum robot, wherein the control means includes a safe mode that disengages the driver from the at least one wire.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a block diagram of an exemplary bendable medical device incorporating various ancillary components, according to one or more embodiment of the subject apparatus, method or system.

FIG. 2 illustrates a kinematic model of the subject continuum robot, according to one or more embodiment of the subject apparatus, method or system.

FIG. 3 provides a detailed illustration of the subject continuum robot, according to one or more embodiment of the subject apparatus, method or system.

FIG. 4 is a top perspective view of the subject continuum robot, according to one or more embodiment of the subject apparatus, method or system.

FIG. 5 illustrates an exemplary solenoid connector according to one or more embodiment of the subject apparatus, method or system.

FIG. 6 is a top perspective view of the subject continuum robot and actuation unit, according to one or more embodiment of the subject apparatus, method or system.

FIGS. 7A and 7B are top perspective views of the subject continuum robot and actuation unit, with FIG. 7B providing a close-up internal view, according to one or more embodiment of the subject apparatus, method or system.

FIG. 8 is a cut-away top perspective view of the subject continuum robot and actuation unit, according to one or more embodiment of the subject apparatus, method or system.

FIGS. 9A and 9B provide a top perspective view (9A) and internal view of the actuator drivers, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In the subject disclosure, Applicant will first detail the mechanism of a continuum robot, followed by the continuum robot attachment/detachment functionality methods, as well as the systems and procedures associated with the continuum robot and said functionality.

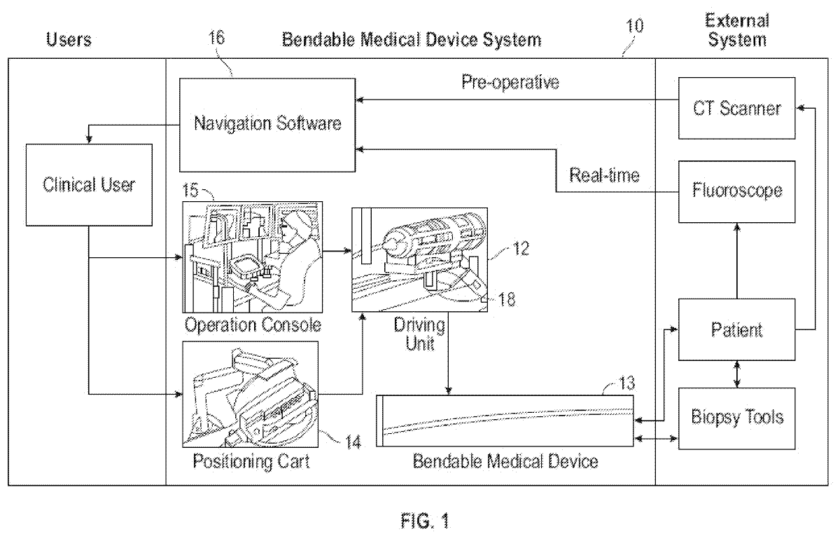

FIG. 1 is a system block diagram of an exemplary bendable medical device system 10 incorporating various ancillary components intended to amass a complete medical system. The bendable medical device system 10 comprises an actuator or driving unit 12 (also referred to herein as a ‘driver’) for driving the wires, and having a base stage 18 (also referred to herein as a ‘base stage 18’), a bendable medical device 13, a positioning cart 14, an operation console 15, having push-button, thumbstick, and/or joystick operational console 15, and navigation software 16. The exemplary bendable medical device system 10 is capable of interacting with external system component and clinical users to facilitate use in a patient.

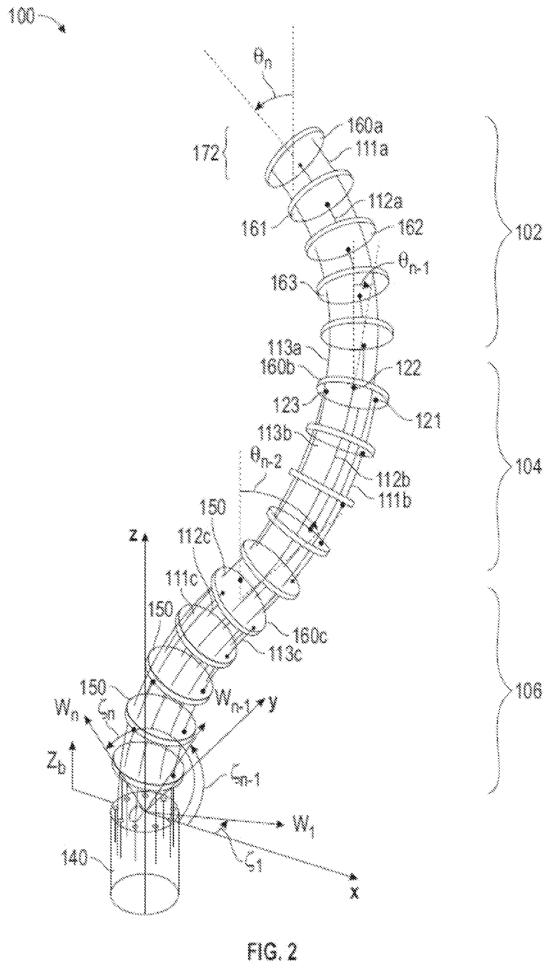

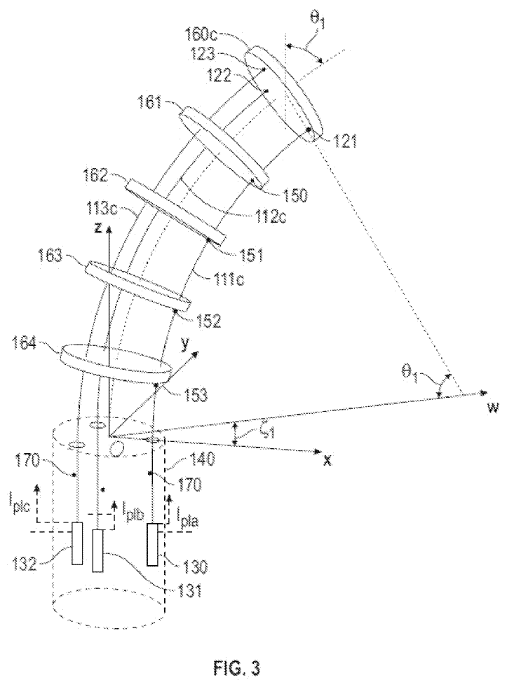

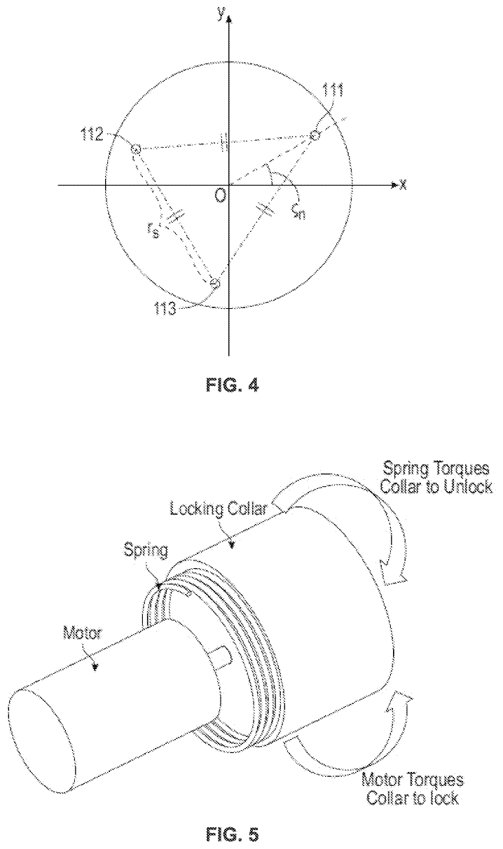

FIG. 2 illustrates a continuum robot 100 that is capable of a plurality of bends, with FIG. 3 providing an enlarged view of a proximal bending section 106 at the proximal end of the robot 100.

As shown in FIG. 2, the continuum robot 100, comprises wires 111b, 112b and 113b, which are connected to connection portions 121, 122 and 123, respectively, found on an end disc 160b, for controlling the middle bending section 104. Additional wires (3 for each of the other bendable sections 102 and 106) 111a, 111c, 112a, 112c, 113a, 113c, are attached at the distal ends of each bendable section 102 and 106, to the respective end disc 160a and 160c.

As each bending section is operated similarly, we will focus on one bending section, here the middle bending section 104, to explain the mechanism. The posture of the bending section 104 is controlled by pushing and pulling the wires 111b to 113b by using actuators 130 to 132 disposed in a robot base 140. (Note—In the interest of clarity, only actuators for the three wires 111c, 112c, 113c have been show in FIG. 3, additional actuators for the remaining 6 wires are contemplated in this innovation.)

Moreover, the robot base 140 of the continuum robot 100 is disposed on a base stage 18 (See FIG. 1) and can be moved by the base stage 18 in the longitudinal direction. Thus, it is possible to advance and retard the robot 100 into a target structure by advancing and retarding the base stage 18.

An operational console 15 (see FIG. 1) indicates a driving amount to the base stage 18 and, independently, to the actuators 130 to 132. Throughout this disclosure, the operational console 15 may also be described or eluded to as a control system or controller. The operational console 15 may include dedicated hardware including a field-programmable gate array (“FPGA”) and the like; or may be a computer including a storage unit, a work memory, and a central processing unit (“CPU”). In the case where the operational console 15 is a computer, the storage unit may store a software program corresponding to an algorithm of the control system (described below) and the central processing unit expands the program in the work memory, executes the program line by line, and thereby the computer functions as the operational console 15. In either case, the operational console 15 is communicably connected to the base stage 18 and the actuators 130 to 132, and the operational console 15 send signals representing the driving amount and configuration to these control targets, which are imputed by an end user through push buttons, joystick or the like.

The continuum robot 100 includes multiple wire guides 161 to 164 situated longitudinally at a distance from one another, throughout each bending section, and moreover detailed in FIG. 3 for proximal bending section 106. The wire guides 161 to 164 are shown here guiding the wires 111c, 112c and 113c, and for providing structural integrity to the bending section 106. As before and for the sake of redundancy, we have elected to detail the components of the proximal bending section 106 in FIG. 3, with the understanding that the remaining bending sections 102 and 104, function in a similar fashion with similar elements. The wire guides 161 to 164 each contain a wire through 150-153 for each wire 111c-113c. For ease of illustration, FIG. 3 only depicts the wire through 150-153 for a single wire 111c. Alternatively, a method of discretely arranging the plurality of wire guides, a continuum robot 100 having a bellows-like shape or a mesh-like shape may be utilized, wherein the wire guides 161-164 are fixed to their respective wires 111a-113a.

Alternatively, a method of discretely arranging the plurality of wire guides 161 to 164, a continuum robot 100 having a bellows-like shape or a mesh-like shape may be utilized, wherein the wire guides 161-164 are fixed to their respective wires 111-113.

With respect to FIGS. 2 and 3, the definitions of symbols are as follows: I_{d}=the length of the central axis a bending section; θₙ=the bending angle of the distal end; ζₙ=the rotational angle of the distal end; ρₙ=the radius of curvature of a bending section.

As seen above, the wires 111-113 may be referred to as wires a, b, and c, counterclockwise in the xy plane; and the driving displacements of the wires for driving the n-th bending section are denoted by Iₚₙₐ, I_{pnb}, and I_{pnc}. As illustrated in FIG. 5, the wires 111-113 are disposed at the vertices of an equilateral triangle whose side has a length rₛ. The phase angle ξₙ is an angle that determines the wire arrangement for driving the n-th bending section. In the present embodiment, ξ₁=0.

### Continuum Robot Attachment/Detachment

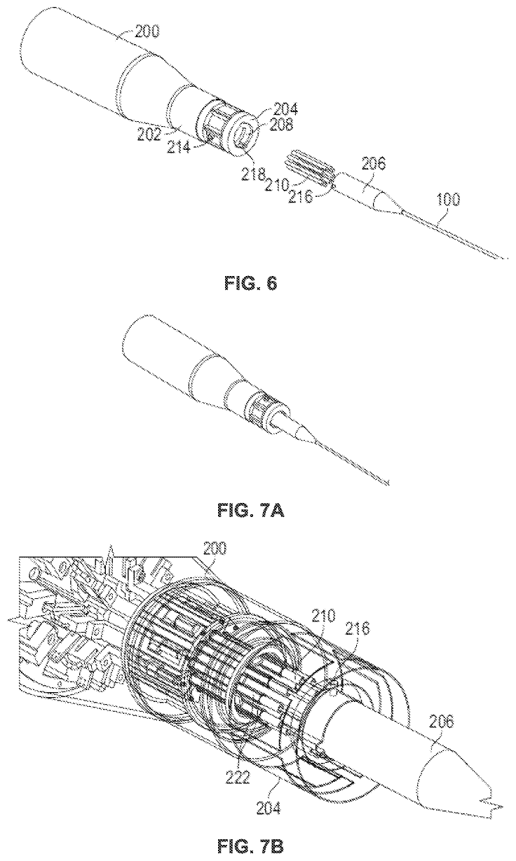

The subject continuum robot 100 utilizes two connection interfaces to attach the robot 100 to the actuator 200. The first connection interface (also referred to as the “body connector interface”) is detailed in FIG. 6, wherein the continuum robot 100 is removably matted to the actuator body 202 using a rotating locking collar 204. This is achieved by inserting the catheter hub 206 into the actuator cavity 208. Once inserted the user rotates the locking collar 204 clockwise which secures the catheter hub 206 in place by engaging protrusions 210 on the catheter hub 206 into respective slotted cavities 212 in the locking collar 204.

The first connection interface between the robot 100 and actuator 200, may be disconnected by rotating the rotating locking collar 204, and separating the catheter hub 206 from the actuator cavity 208. As seen in FIGS. 6 and 7B, rotating the locking collar 204, disengages the protrusions 210 on the catheter hub 206 from the respective slotted cavities 212, allowing for separation. The locking collar 204 may be urged is a clockwise or counter-clockwise rotation by incorporating an optional spring 218 (see FIG. 5). The optional spring 218 may be beneficial in automating the rotation step for an end user, which may further allow for engagement/disengagement of the locking collar 204 by one hand rather than two.

As further seen in FIGS. 6, 7A and 7B, a push button 214 and slot 218 may be used on the locking collar 204 as a method for securing the catheter hub 206 to the actuator 200, such that the rotating locking collar 204 is not inadvertently rotated and disconnected. The slot 218 aligns with a key 216 found on the catheter hub 206, to ensure accurate insertion of the catheter hub 206 into the locking collar 204, and further acts to lock the locking collar 204 with the catheter hub 206, once the locking collar 204 is rotated. In addition, the push button may be required to be compressed to allow the locking collar 204 to be rotated, thus allowing the dislocation of the actuator 200 from the robot 100.

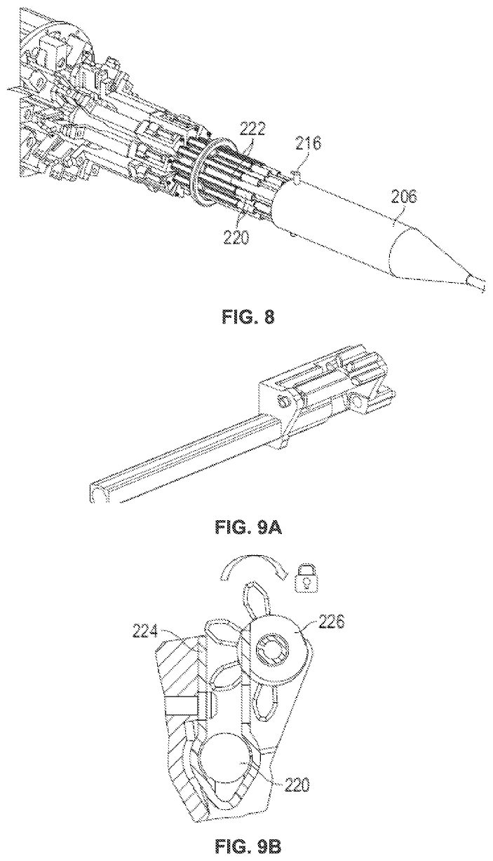

The second connection interface (also referred to as “wire connector interface”) is depicted in FIG. 8, wherein the outer cover of the locking collar 204 has been removed to show the details of the catheter drive wires 220. Here the second connection interface attaches the catheter drive wire(s) 220 to the actuator driver 222. In this embodiment, nine drive wires 220 and their respective actuator drivers 222 are depicted, but as can be appreciated with some one of skill in the art, additional or less drive wires may be utilized based on application and degree of bendability for the robot 100.

Each drive wire 220 is attached to the respective actuator driver 222 when the end user rotates the locking collar 204. As seen in FIGS. 9A and 9B, when the user rotates the locking collar 204 the drive wires are disengaged from the clamp 224. This is accomplished by the cam mechanism 226, which is rotated by the locking collar 204, and urges the clamp 224 together to trap the drive wire 220. The inner surface of the locking collar 204 has teeth that mate with the cam mechanism 226, which in turn rotate the cam mechanism 224, when the locking collar is rotated.

The locking collar 204 is limited in the amount of rotation by the button 214, and thus will not fully unlock keeping the catheter hub 206 in place. This is used is the emergency release mode. When the button 214 is held down the locking collar 204 can be fully rotated and the hub is unlocked allowing the catheter 100 to be removed.

The two connection mechanisms may include sensors to detect attachment/detachment of both interfaces. In this embodiment, these interfaces form a continuum robot-to-actuator connector and have the following operation sequence by its mechanical design:

Catheter Attachment—When the physician operates the catheter 100 to actuator 200 connector to attach the catheter 100 with the actuation unit, the body connector interface is mechanically activated and attaches the continuum robot 100, then the wire actuator drivers 222 are mechanically activated and attach to the driving wires 220. In another example, these activation and attachments in the body connector interface and wire connector interface may happen at the same time.

Catheter Detachment—When the physician operates the catheter 100 to actuator 200 connector to detach the catheter from the actuation unit, the wire actuator drivers 222 are mechanically activated and detach the driving wires 220, then also, the body connector interface is mechanically activated and detaches the continuum robot 100. Furthermore, in another example, these activation and detachments in the body connector interface and wire connector interface happen at the same time.

A key technology for the catheter attachment and detachment as a normal operation is to have a system indicator for attachment of the continuum robot 100 to the actuator body and attachment of the drive wires 220 to the motorized linear mechanisms. This can be integrated through separate sensors that track the connections in the body connection interface and the wire connection interface individually. If one of these signals does not show a positive value for connection, then the user cannot proceed to operational mode and will be promoted to complete the connection and/or troubleshoot. An additional way to track the connection in the wire connection interface is through force sensor reading. If no force reading is shown when slight motion is induced, then the system can detect an issue with wire connection.

For removal of the catheter 100, the workflow is similar, however in reverse. First the user will be prompted to disengage the drive wires 220, however leave the hub 204 connected. Once this condition is satisfied the system will prompt the user to reverse the base stage 18 to a point where removal is safe. Once this condition is satisfied the user will be prompted to remove the catheter hub 204 from the actuator 200. Once this is completed and verified through the sensors, power down shall be allowable.

In another exemplary design, the system can have the specific position range of the base stage to allow the physician executing the catheter attachment/detachment as the normal operation. The base stage includes a position sensor for the system to detect the base stage position. After the system is powered on, the base stage is set to this position range. At this point, the system enters Attachment/Detachment mode (detailed above). In this mode, the physician can attach or detach the catheter to the actuation unit as the normal operation. By combining the position sensor in the base stage with the sensors in the body connection interface and wire connection interface, the system can distinguish the normal attachment/detachment from the irregular attachment/detachment.

In another exemplary design, the Attachment/Detachment Mode allows for a workflow that transitions from power on to operational mode (attachment) or operational mode to power down (detachment). There is also the case where the catheter may be attached before power up. The system shall be capable of recognizing this connection on startup. For removal, the workflow is similar, however in reverse. First the user will be prompted to disengage the drive wires, however, leave the hub connected. Once this condition is satisfied, the system will prompt the user to reverse the linear stage to a point where removal is safe. Once this condition is satisfied the user is prompted to remove the catheter hub from the actuator, which is verified through the sensors, and power down is allowable (given other procedures have been prompted outside the scope of this MOI). There is also the case where the catheter may be attached before power up. The system shall be capable of recognizing this connection on startup.

### Safe Hold Mode

The robotic steerable catheter system includes at least one Safe hold mode besides a normal operation mode. In the Safe hold mode, the system will stop any obtrusive operation. For example, in this embodiment, the system safely holds the positions of the motorized linear motion mechanisms in the actuation unit, as well as the linear motor in the base stage, when the system state enters Safe hold mode. In addition, the robot controller won't accept the operation commands from the operator except for defined limited commands until the system state exits Safe hold mode. The system won't quit from Safe hold mode until the physician confirms safe conditions, and commands to quit Safe hold mode.

### Catheter Emergency Detachment

The catheter-to-actuator connector in this disclosure includes the following unique emergency detachment. When the physician operates the catheter-to-actuator connector to detach the steerable catheter with this emergency detachment, the wire connector interfaces are mechanically activated and detach the driving wires. However, the body connector interface is NOT mechanically effected, and the body of the catheter remains connected with the body of the actuation unit.

With this catheter emergency detachment method, the physician can stop bending the catheter and make the catheter flexible by disengaging the driving wires from the actuation unit immediately and with safety. By allowing the body of the catheter to remain attached with the body of the actuation unit, the physician cannot associate further hazardous situation, e.g., dropping the catheter upon the patient or further adverse interaction between the catheter and the patient.

In another design example, the catheter-to-actuator connector can include this catheter emergency detachment in the middle of the catheter detachment sequence. In this design, to remove the catheter, when the physician operates the catheter-to-actuator connector, first, the wire connector interfaces are mechanically activated and the driving wires are detached. However, without an additional button action, the catheter-to-actuator connector will not activate the body connector interface and will not disconnect the body of the catheter from the body of the actuation unit (equivalent to the completion of the catheter emergency detachment). When the physician detaches the continuum robot 100 from the actuation unit as the normal removal operation, the physician can perform the additional button action, and activate the body connector interface to detach the body of the catheter. Therefore, the catheter is detached via the catheter emergency detachment.

With this design, the physician can access the emergency detachment as a priority with minimal operational burden and duration of time in engaging the emergency detachment. In addition, this can reduce any possible confusion a physician may have during an emergency between the normal detachment operation and the operation for the catheter emergency detachment, since the physician remembers only one detachment operation including the catheter emergency detachment.

This system can distinguish the Safe hold mode from standard attachment/removal mode by receiving an external signal from patient monitoring system or from the user input (i.e. e-stop button or GUI input).

### Embodiment #1

In the first exemplary embodiment, there are three states. The first state is in normal operation state, which can be any of the Snake operating modes (FTL, rFTL, Target, BKD mode). In this state, if an issue arises in which an indication of the requirement of emergency removal occurs (which includes an electrical signal from the patient monitoring system, a visual indication to the physician/user, or other indicators) the user pushes a stop button, or the monitor sends a signal to the snake system, which allows the system to enter a second state. The second state is a “transition to emergency removal mode”, where the snake system is aware of the need to enter emergency removal mode, however, the snake system requires certain action(s) to be carried out to enter that state. In this case the user may twists the connector (between the catheter and actuator), which disengages the wire allowing the catheter tip to become flexible and safe for removal. This action notifies the Snake system, to enter the third state, wherein the user has disconnected the catheter and now allows functionality to remove the catheter. To elaborate, the third state will notify the software to allow the end user to pull the base stage backward to remove the catheter from the patient, while still restricting forward motion.

To further elaborate on the functionality restrictions, in the transition state (second state) the base stage would not be able to move in either direction due to the drive wire being engaged and potentially harmful to the patient if attempted to be removed. Once in the third state, the base stage is free to remove the catheter. In both the transition and emergency state all motion to the catheter drive wire would be restricted.

### Embodiment #2

While the overall functionality of the second system embodiment is roughly the same as the first embodiment, in the second embodiment there are only two states, as the 2^{nd} and 3^{rd} state from the first embodiment are combined.

Accordingly, when the catheter emergency detachment happens, the system can detect the catheter emergency detachment with the sensors in the wire connector interface and the body connector interface and moves to the Safe hold mode automatically. This interlocking with the catheter emergency detachment allows reduced risks of further hazardous situations without any physician's enactment.

Specifically, since the Safe hold mode happens when the driving wires are detached, but the body of the catheter is still connected with the actuation unit, the physician can safely remove the catheter from the patient without the catheter coming loose.

This second embodiment has an advantage as there are fewer steps that need to be carried out by the user, and therefore less chance of human error. Since the wire disengagement is the necessary first step to remove the robotic catheter from the patient in any situation, the physician can just remember they need to disengage the driving wires before removing the catheter from the patient manually.

Also, the physician can use this automatic entering the safe hold mode with disengagement of the driving wires intentionally for the emergency situation.

To elaborate, the action of the user twisting the connector automatically sends a signal to the Snake system allowing it to enter catheter emergency detachment mode. For this to work a sensor would be imbedded on the actuator side of the connector. When the catheter is twisted, the sensor trips. This removes a lot of the user confusion and error that can occur in the transition state from the first embodiment, however this requires a slightly more complex design.

### Embodiment #3

This embodiment has the same workflow as the second embodiment, however instead of relying on the user to twist the connector to enter the emergency state, a built-in mechanism will be triggered which automatically disengages the wire. In one example, Safe hold mode would be triggered by the user hitting a button (physical or software), or an input from a patient monitoring system.

As seen in FIG. 5, the mechanism would work like a spring solenoid that in a power on state, applies force against the spring, and in a power off state, the spring expands. For the Snake system a torsional spring could be utilized to force the catheter connector to naturally go to an unlocked position where all the drive wires are free. However, when the system is powered on and set to an operational mode, a motor turns on and applies a torsional force that twists the catheter connector into a locked position. When Safe hold mode is engaged, the motor is powered off and the catheter wire automatically disconnects.

An advantage of this method is it also automatically disengages the wire in a power failure situation.

### Embodiment #4

While the overall functionality of System Embodiment #4 is roughly the same as System Embodiment #2, the system in this embodiment includes the manual insertion operation with the manual slide stage.

The manual slide stage is mounted on the base stage 18 and has the actuation unit.

Therefore, the robotic catheter on the actuation unit can be inserted or removed by moving either the manual slide stage or the base stage. Besides a position sensor in the base stage, this manual slide stage also includes secondary position sensor.

Before powering on the system, the robotic catheter is not attached to the actuation unit. After powering the system on, the system sets the base stage position to the start position in the position range of the normal catheter attachment/detachment. Also, the system instructs the operator to set the manual slide stage to the position for the normal catheter attachment/detachment. At this point, the system enters Attachment/Detachment mode.

During the attachment/detachment mode, the operator attaches the robotic catheter by interacting with the catheter-to-actuator connector.

After attaching the robotic catheter on the actuation unit, the physician instructs the robotic controller to quit the attachment/detachment mode. Then, the physician inserts the robotic catheter from the patient mouse to carina by sliding the manual slide stage manually. After this manual insertion step, the physician locks the position of the manual slide stage (manual insertion) and continue to insert the robotic catheter using the robotic steering control with the base stage by using the joystick (robotic insertion).

During the robotic insertion, if the manual slide stage is moved (intentionally, accidentally or mistakenly) from the position of the manual slide stage at the beginning of the robotic insertion, the system automatically enters the safe hold mode.

Also, in another example design, the manual slide stage includes a sensor to detect lock/unlock of the manual slide stage. If the unlock is detected during the robotic insertion (intentionally, accidentally or mistakenly), the system automatically enters the safe hold mode.

By automatically entering the safe hold mode with any movement of manual slide stage or the unlocking of the stage, the system can avoid an irregular robotic insertion with unintended offset insertion position of the robotic catheter.

### Embodiment #5

While the overall functionality of System Embodiment #5 is substantially the same as System Embodiment #2, the system in this embodiment includes an actuation unit detachably attached on the base stage.

The base stage has a sensor to detect the attachment/detachment of the actuation unit. The system enters attachment/detachment mode only when the actuation unit is attached to the base stage in the specific workflow procedure that the robot controller manages, e.g., a setup step.

During robotic insertion, if the actuation unit is detached (intentionally, accidentally or mistakenly) from the base stage, the system automatically enters the safe hold mode. By automatically entering the safe hold mode with the detachment of the actuation unit, the system can avoid further hazardous situation after detaching the actuation unit.

Furthermore, it is contemplated that although the catheter is typically removed from the actuator in normal operation, during emergency mode the actuator can be removed from the base stage (where the insertion state is located) in order to remove the distal catheter body from the subject. The advantage to this as opposed to removing the catheter is that you don't have to create forward motion. When you remove the catheter from the actuator, you have to insert the catheter further into the patient which could be dangerous, to disengage from the actuator. This workflow is similar to manually sliding the Z stage positioner to the rear position.

1. A robotic apparatus comprising:
a continuum robot including a first bending section having a distal end and a proximal end wherein the first bending section is bent by at least one wire;
a driver that drives the at least one wire; and
a controller that controls a driving amount of the wire,
wherein, the driver further comprises a safe mode that disengages the driver from the at least one wire.

2. The apparatus for claim 1, further comprising a mechanical locking collar configured on the driver for engaging or disengaging the driver from the at least one wire.

3. The apparatus of claim 2, wherein the locking collar retains a mechanical connection with the first bending section while disengaging the driver from the at least one wire.

4. The apparatus for claim 1, further comprising a second bending section bent by a second wire.

5. The apparatus for claim 4, wherein the controller controls a driving amount of the second wire.

6. The apparatus for claim 1, further comprising a base stage attached to the controller for advancing and retracting the controller and attached continuum robot.

7. A robotic apparatus comprising:
a continuum robot including a bending section wherein the bending section is bent by at least one wire;
a driver that drives the wire;
a base stage affixed to the continuum robot and capable of moving the continuum robot;
a controller that is in electrical communication with the driver and the base stage, and controls a driving amount of the wire and the base stage; and
a locking collar configured on the driver for engaging or disengaging the driver to the at least one wire,
wherein, the controller further comprises a safe mode that restricts the driver from driving the at least one wire.

8. The apparatus of claim 7, wherein the locking collar retains a mechanical connection with the first bending section while disengaging the driver from the at least one wire.

9. The apparatus for claim 7, further comprising a second bending section bent by a second wire.

10. The apparatus for claim 9, wherein the controller controls a driving amount of the second wire.

11. Method of operating a continuum robot comprising:
a continuum robot including a first bending section having a distal end and a proximal end wherein the first bending section is bent by at least one wire;
the method comprising:
driving the at least one wire by the driver; and
controlling a driving amount of the wire by the controller, to enable a bending angle and a rotational angle of the first bending section,
wherein the controller includes a safe mode that prohibits the driver from driving the at least one wire.

12. The method for claim 11, further comprising a locking collar configured on the driver for engaging or disengaging the driver from the at least one wire.

13. The method of claim 12, wherein the locking collar retains a mechanical connection with the first bending section while disengaging the driver from the at least one wire.

14. The apparatus for claim 11, further comprising a second bending section bent by a second wire.

15. The apparatus for claim 14, wherein the controller controls a driving amount of the second wire.

16. The apparatus for claim 11, further comprising a base stage attached to the controller for advancing and retracting the controller and attached continuum robot.

17. Continuum robot control means comprising:
a continuum robot including a plurality of bending sections including a distal bending section and a proximal bending section wherein each of the bending sections are driven by at least one wire;
driving means that drives the wire;
control means that controls a wire driving amount from a bending angle and a rotational angle of the continuum robot, and the control means controls a base affixed to the continuum robot; and
wherein the control means includes a safe mode that disengages the driver from the at least one wire.
